(19) United States
(12) Patent Application Publication
(10) Pub. No.: US 2009/0281391 A1
(43) Pub. Date: Nov. 12, 2009

(54) DISSECTION-ASSISTING RETRACTOR

(76) Inventor: Michael D. Baskett, Centreville, VA (US)

Correspondence Address: SHAWNA J. SHAW, 15151 STRATTON MAJOR CT., CENTERVILLE, VA, 20120, US

(21) Appl. No.: 12/119,404

(22) Filed: May 12, 2008

(51) Int. Cl. A61B 1/32

(52) U.S. Cl. 600/201

(57) ABSTRACT

A surgical retractor for assisting in dissection procedures including a main housing, a handle coupled to a proximal portion of the main housing, and one or more movable belts coupled to a distal portion of the main housing. Preferably, the one or more belts are adapted to frictionally engage and retract tissue from a surgical site. In embodiments, one or more of the belts are comprised of a material with sufficient friction properties to securely engage tissue. In some embodiments, one or more of the belts also have a plurality of grooves for securely engaging tissue. In further embodiments, the main housing also includes a distal lip for further engaging tissue in the incision site. Preferably, the retractor is able to gently pull tissue away from a surgical site without injuring or damaging surrounding healthy tissue.

### BACKGROUND OF THE DISCLOSURE

1. Field of the Disclosure

The present disclosure relates generally to surgical instruments. More specifically, the present disclosure relates to a surgical retractor.

2. Background of the Disclosure

Surgical retractors are generally used to separate the edges of an incision so that the regions under the incision may be accessed while maintaining a clear surgical site. For example in dissection procedures, retractors are inserted into the incision and used to hold layers of tissue out of the way so that accurate dissections may be made. As used herein, “tissue” includes various types of tissues including skin, fat, fascia, muscle, etc.

One common problem with existing retractors includes slipping of tissue. For example, in shallow incisions it is difficult to maintain a good grasp along the edge of the incision and therefore to hold the retractor in place. In other procedures, such as forearm fractures, large oppositional forces exerted e.g., by muscle tissue in the incision site may increase tendencies for retractors to slip. As a result of such slipping or shifting, retractors may need to be frequently repositioned. However, repositioning unnecessarily slows down the procedure and hinders surgical accuracy.

Well-known retractors include: Richardson retractors, Weitlaner retractors, Gelpi retractors, Henderson retractors, Army-Navy retractors, Deaver retractors, and the like. For example, Richardson-type retractors typically have a smooth, right angle blade to hold tissue away from a surgical site. The blade is inserted into the incision and used to pull back tissues in a “hoe-like” manner. However, such retractors have tendencies to slip from desired positions (particularly when used for shallow incisions), and to put strain on the operator when used to hold back strong tissues such as muscle. Moreover, because these types of retractors are used to exert direct “pulling” forces along the edges of the incision, tissues next to the incision are “bunched” together resulting in uneven surfaces.

Other retractors include teeth or hooks to engage or penetrate tissue adjacent an incision and prevent slipping. For example, Weitlaner retractors are a type of self-locking retractor typically comprising two and three, or three and four, teeth at the distal end. However, such teeth or hooks may injure or damage surrounding healthy tissue thereby increasing risks of infection and healing time.

Thus, there exists a need in the art for a surgical retractor for assisting in dissection procedures that provides improved tissue engaging capabilities with minimal injury to surrounding healthy tissue. There is also a need in the art for a surgical retractor that is able to maintain a flat or even tissue surface at a surgical site for making incisions. Furthermore, there is a need to provide a surgical retractor that lessens the physical strain on the operator and is easy to use.

### SUMMARY OF THE DISCLOSURE

The present disclosure overcomes drawbacks of the prior art by providing a surgical retractor e.g., for assisting in dissection procedures that provides improved tissue engaging capabilities for holding tissue away from a surgical site with minimal damage or injury. Such tissue engaging capabilities are enabled by one or more movable belts designed to gently yet securely grasp tissue adjacent to an incision. The present disclosure also overcomes drawbacks of the prior art by providing one or more movable belts that are able to gently apply a continuous opposing force to tissue to maintain a flat, even surface for making incisions. The present disclosure further overcomes drawbacks of the prior art by providing a surgical retractor with belts that are driven by one or more drive assemblies requiring minimal effort on the part of an operator.

Unlike retractors currently in use which primarily pull or push on tissues along the edge of an incision and/or use teeth or hooks to engage tissue, the surgical retractor of the present disclosure is able to hold adjacent tissues out of the way of an incision using surface traction. Thus, unlike prior art retractors which may require large amounts of pulling forces to be applied directly to the edge of an incision (increasing risks of tearing), the retractor disclosed herein is able to distribute forces evenly over the surface of adjacent tissue. As a result, less direct forces need to be applied to edge of the incision—reducing risks of tearing tissue as well as operator strain.

According to one aspect, the disclosure provides a surgical retractor, the retractor comprising: a main housing; a handle coupled to a proximal portion of the main housing; a first belt coupled to a distal portion of the main housing and movable by a primary drive assembly; one or more additional belts coupled to a distal portion of the main housing, the one or more additional belts movable by a secondary drive assembly; and wherein the one or more additional movable belts are adapted to frictionally engage and retract tissue(s) from a surgical site. Preferably, one or more of the belts are comprised of a material with sufficient friction properties to securely engage tissue without slipping. In embodiments, one or more of the additional belts have a plurality of grooves for further securely grasping tissue. In further embodiments, the main housing includes a distal lip for additionally engaging tissue within the incision.

According to another aspect, the disclosure provides a surgical retractor, the retractor comprising: a main housing; a handle coupled to a proximal portion of the main housing; and one or more movable belts coupled to a distal portion of the main housing and adapted to frictionally engage and retract tissue(s) from a surgical site. Preferably, one or more of the belts are comprised of a material with sufficient friction properties to securely engage tissue without slipping. In embodiments, one or more of the additional belts have a plurality of grooves for further securely grasping tissue. In further embodiments, the main housing includes a distal lip for additionally engaging tissue within the incision.

According to a further aspect, the disclosure provides a method for retracting tissue, the method comprising: providing a surgical retractor, the retractor including a main housing, a handle coupled to a proximal portion of the main housing, and one or more movable belts coupled to a distal portion of the main housing and adapted to frictionally engage tissue(s) adjacent to a surgical site; and retracting the tissue(s) by moving the one or more of the movable belts. In preferred embodiments, the one or more belts are moved manually.

One advantage of the disclosed surgical retractor is its ability to securely hold adjacent tissue away from an incision site without damage or injury. For example, by using traction to engage adjacent tissue rather than teeth, hooks, or blades—damage to surrounding healthy tissue is significantly reduced.

Another advantage of the disclosed device is the ability to gently pull adjacent tissue away from an incision site in a continuous manner. As a result, a flat, even tissue surface may be maintained while an incision is performed.

Yet another advantage is that the need for repositioning is minimized and overall procedure time reduced.

A further advantage of the disclosed device is the ability to reduce the amount of direct “pulling” forces applied to an incision. For example, because traction forces may be evenly distributed adjacent to the incision, direct forces applied to the edges of the incision are reduced (thereby also reducing chances of tearing tissue). In addition, the tissue engaging belts are driven by one or more drive assemblies which are able to mechanically reduce the amount of force required to be applied by the operator.

### BRIEF DESCRIPTION OF THE DRAWINGS

FIG. 1 shows an isometric view of a surgical retractor according to a preferred embodiment of the present disclosure.

FIG. 2 shows a top view of a surgical retractor according to a preferred embodiment of the present disclosure.

FIG. 3 shows a cut-away side view of a surgical retractor according to a preferred embodiment of the present disclosure.

FIG. 4 shows a detailed cut-away view of a secondary drive mechanism according to an exemplary embodiment of the present disclosure.

FIG. 5 shows a component view of a first belt according to an exemplary embodiment of the present disclosure.

### DETAILED DESCRIPTION OF VARIOUS EMBODIMENTS OF THE DISCLOSURE

Reference will now be made in detail to various exemplary embodiments of the disclosure, examples of which are illustrated in the accompanying drawings. The following detailed description describes certain embodiments of the disclosure, and should not be considered as limiting the disclosure to those embodiments.

The present disclosure offers a solution to problems associated with slipping or shifting of tissue during incision or dissection procedures. The solution is a retractor including one or more tissue engaging belts that securely hold tissue away from a surgical site with minimal injury or damage to adjacent healthy tissue. The present disclosure thus provides improved tissue grasping and pulling capabilities. Of the many other advantages provided by the device, it is simple and easy to use, improves performance of dissection procedures, reduces risk of injury to the patient, and saves repositioning time.

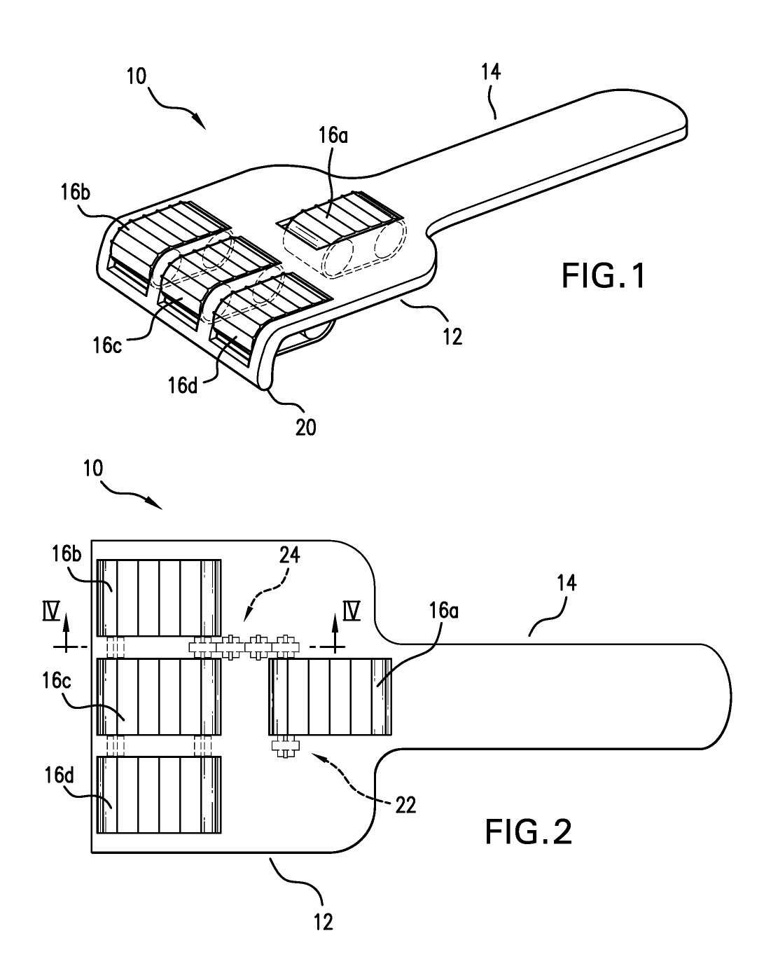

Turning now to the figures, which depict various exemplary embodiments of the disclosure, a surgical retractor is shown that securely engages tissue e.g., for dissection procedures. FIG. 1 shows an isometric view of the surgical retractor (generally depicted as element 10) according to a preferred embodiment. The surgical retractor 10 comprises a main housing 12 having a proximal and a distal end, as well as a top side and a bottom side. The proximal end comprises a handle 14 for an operator and the distal end includes a lip 20 for engaging tissue along the edge of an incision. Further shown on the distal end of the housing 12 are a first belt 16a and one or more additional belts 16b-d. Preferably the belts 16a-d are exposed on the bottom side of the housing 12 to securely engage tissue adjacent to the incision. It is appreciated that some of belts 16 may be exposed on the top side of housing 12, or alternatively may be covered by housing 12. In the embodiment shown, some of the belts 16 include grooves 18 thereon to further aid in making secure contact with tissue. However, it is understood that in other embodiments, different mechanisms for securely engaging tissue may be used. Moreover, while four belts 16a-d are illustrated by way of example, other quantities of belts 16 may be provided depending upon the application the retractor 10 is to be used for.

It is appreciated that housing 12 may be comprised of metal, stainless steel, plastic, or any other material suitable to perform the functions disclosed herein. Moreover, the size and shape of the housing 12 may be varied depending upon the type of surgery or application of use. For example, the housing 12 may be larger for abdominal surgeries, and smaller for minor dermatological procedures, etc.

In addition, the handle 14 may be integral to the housing 12 or detachable. For example, the handle 14 may be attached to the housing 12 by means of a modified dovetail joint, pins, screws, and the like. It is also understood that the handle 14 may have various shapes depending upon its intended application and is therefore not limited to the embodiment shown. It is also possible that handle 14 may be designed with ergonomic considerations in mind.

In operation, the belts 16b-d serve to engage tissue with a small amount of contact force applied by an operator and to gently pull tissue away from the surgical site via sufficient amounts of friction forces. By using a plurality of belts 16a-d, tissue is also caused to spread out in a more even manner. Preferably, belts 16a-d are comprised of a material having sufficient friction properties for securely engaging tissue and gently pulling the tissue away from the surgical site. Examples of suitable belt materials include rubber-type materials such as silastic™ (by Dow Corning, Inc.), etc. Silastic™ materials advantageously have a coefficient of friction that can be modified depending upon the application. In addition, certain silastic™ materials are well suited for surgical applications because they are silicone based and can be easily sterilized using conventional sterilization techniques such as high temperatures, high pressures, sonication, UV radiation, etc.

In embodiments, the belts may include a plurality of grooves 18 for securely engaging tissue. Such grooves 18 may be molded into the belts 16b-d or integrated in another equivalent manner. In addition, the grooves 18 may assume various cross-sectional configurations for engaging tissue such as triangular, saw-shaped, dome-shaped, square-shaped, and the like. Moreover, the size of the grooves 18 may vary depending upon the particular application and required traction forces.

In embodiments, the dimensions of the lip 20 may vary depending upon the type of surgery. For example, a deeper lip 20 may be used for abdominal surgeries. Preferably, the lip 20 does not have any sharp protrusions or blades which could cause tissue damage. Alternatively, it is also within the scope of the disclosure to provide a retractor 10 without a lip 20. Such an embodiment may be beneficial for shallow incisions e.g., such as dermatological procedures, etc. Advantageously, smaller incisions could be made that would not require the retractor 10 to be placed into the incision.

FIG. 2 illustrates a top view of the surgical retractor 10. The movable belts 16a-d are shown at the distal end of the housing 12 and handle 14 is shown at the proximal end. Also depicted in FIG. 2 are a primary drive assembly 22 and a secondary drive assembly 24. Preferably, the first belt 16a is movable in a conveyor belt-type manner by the primary drive assembly 22, and belts 16b-d are movable in a conveyor belt-type manner by the secondary drive assembly 24. In preferred embodiments, the secondary drive assembly 24 is driven by the primary drive assembly 22 to maintain movement of the belts in a synchronous fashion. It is further appreciated that the primary and secondary drive assemblies 22 and 24 may be a gear assembly, or other equivalent mechanism for moving the belts in a synchronous 16a-d manner. Additionally, a conventional locking mechanism (not shown) may be used to lock the drive assemblies 22 and 24 in place as will be appreciated by those skilled in the art.

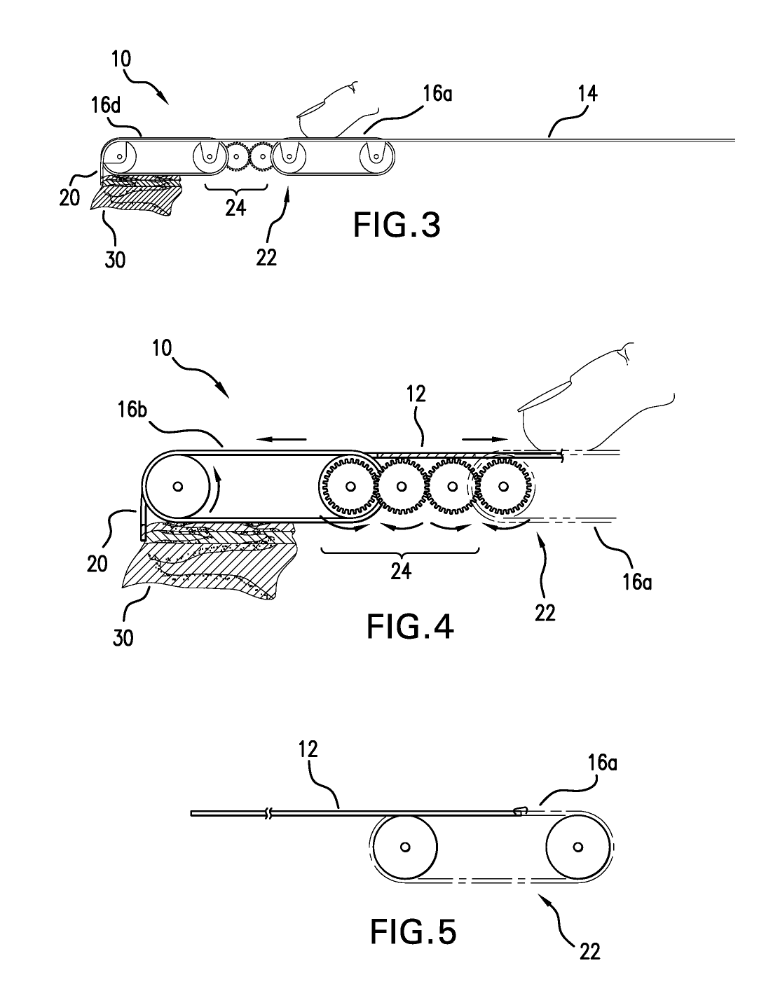

FIG. 3 illustrates a side view of the surgical retractor 10 as cut-away along line IV-IV (see FIG. 2). As shown the figure, first belt 16a is coupled to belt 16d via secondary drive mechanism 24. Thus, movement of belt 16a (e.g., by finger-movement of an operator) causes synchronous movement of belt 16d. As belt 16d engages tissue 30 adjacent to an incision, the tissue 30 is gently and continuously pulled back from the incision site to maintain an even tissue surface. Looking at FIG. 3, the retractor 10 may be held by an operator using handle 14 and operated by moving first belt 16a “clockwise” as shown (e.g., by finger movement). As also illustrated in FIG. 3, lip 20 may engage the edge of an incision further helping to hold the incision open. One difference here from prior art retractors, is that the lip 20 does not apply large “pulling” forces against the edge of the incision, but more passively engages the edge of the incision as surface traction forces are applied to adjacent tissues 30 via belts 16.

FIG. 4 shows a detailed view of the secondary drive assembly 24. Looking at FIG. 4, the retractor may be manually operated e.g., by moving the top portion of belt 16a “away” from the surgical site. Movement of belt 16a in this “clockwise” direction causes belt 16d to correspondingly move in an opposite “counter-clockwise direction” via the secondary drive assembly 24 (which in this embodiment is a simple gear mechanism). Thus, when belt 16d securely engages tissue 30 and is caused to continuously move in a “counter-clockwise” direction, it effects a continuous pulling of adjacent tissue 30 away from the incision. As further shown, lip 20 may engage the edge of the incision to help hold the incision open.

In addition, FIG. 5 shows a component view of belt 16a. As mentioned, primary drive assembly (not shown) and belt 16a may be manually moved by an operator (e.g., by finger movement). Alternatively, it is possible that the primary drive assembly may be automated. However, advantages of manual operation include: simpler design, lower cost, tactile operator feedback, and easier sterilization of the device. For example, manual operation allows the operator to experience force feedback through resistance in belt 16a. Thus by sensing the amount of resistance in belt 16a, the operator may be able to apply only the necessary amounts of horizontal and vertical forces without tearing or damaging tissue.

In embodiments, the entire retractor 10 may be re-used and easily sterilized using conventional sterilization techniques including high temperatures, high pressures, sonication, UV radiation, or the like. Here, the retractor 10 may be comprised of materials such as stainless steel, plastic, rubber, silastic™, etc. In other embodiments, the retractor 10 may be intended for single-use and composed of inexpensive materials including plastic, rubber, etc.

It can be seen from the above discussion that the disclosed retractor provides improved tissue engaging capabilities with minimal injury or damage to healthy tissue. Such tissue engaging capabilities are enabled by one or more belts designed to safely yet securely grasp tissue adjacent an incision and may further be applied in combination with a distal lip to hold tissue away from the surgical site. In addition, the disclosed retractor is able to maintain a flat, even tissue surface for incisions and dissections by gently applying a continuous traction force to adjacent tissue. Moreover, the disclosed device requires minimal effort on the part of an operator, e.g., by providing one or more drive assemblies to move the tissue engaging belts.

By providing one or more movable belts for securely engaging tissue adjacent an incision, the retractor of the present disclosure can securely and gently pull tissue away from an incision site without damaging healthy tissue. As a result, the retractor is able to provide more effective results while additionally saving time from having to reposition the device. In addition, the moving belts provide an advantage not supplied by other retractors because of their ability to securely grasp tissue in a manner that is less prone to slipping and because they do not require teeth or hooks which may damage adjacent healthy tissue.

The foregoing disclosure of the preferred embodiments of the present disclosure has been presented for purposes of illustration and description. It is not intended to be exhaustive or to limit the disclosure to the precise forms disclosed. Many variations and modifications of the embodiments described herein will be apparent to one of ordinary skill in the art in light of the above disclosure. While not limited to any particular use, it is envisioned that the retractor 10 is used to assist in dissection procedures. However, the retractor 10 may be used in other surgical procedures such as excisions, resections, biopsies, abdominal surgeries, neck surgeries, dermatological procedures, and more. Thus, it is further envisioned that the size and shape of the retractor 10 may be varied depending upon the type of surgery or application of use. For example, the retractor 10 may be designed to be larger and wider for leg fractures, and smaller for delicate dermatological procedures. In addition, the shape of the retractor 10 need not be rectangular, but may assume various shapes depending upon the type or surgery or application. For example, the retractor 10 may be narrower at the distal end to help access difficult to reach areas such as ankle fractures. It is also within the scope of the disclosure to provide a retractor 10 without a lip 20. In this case, small incisions may be made without requiring the retractor 10 to be placed into the incision. In addition, while the primary function of the movable belts 16 is to gently “pull” tissue away from a surgical site using traction, it is further possible that the belts 16 may be moved in the opposite direction to gently “push” tissue toward the surgery site, for example to aid in suture procedures, and the like. Accordingly, the scope of the disclosure is to be defined only by the claims appended hereto, and by their equivalents.

1. A surgical retractor, the retractor comprising:
a main housing;
a handle coupled to a proximal portion of the main housing;
a first belt coupled to a distal portion of the main housing and movable by a primary drive assembly;
one or more additional belts coupled to a distal portion of the main housing, the one or more additional belts movable by a secondary drive assembly; and
wherein the one or more additional movable belts are adapted to frictionally engage and retract tissue(s) from a surgical site.

2. The surgical retractor of claim 1, wherein the primary drive assembly is manually operated.

3. The surgical retractor of claim 1, wherein the secondary drive assembly is driven by the primary drive assembly.

4. The surgical retractor of claim 1, wherein one or more belts have a plurality of grooves adapted to securely grasp tissue.

5. The surgical retractor of claim 1, wherein one or more belts are comprised of a rubber-type material.

6. The surgical retractor of claim 1, wherein the handle is detachable from the main housing.

7. The surgical retractor of claim 1, wherein the housing includes a lip at the distal-most portion for engaging tissue along an incision.

8. A surgical retractor, the retractor comprising:
a main housing;
a handle coupled to a proximal portion of the main housing; and
one or more movable belts coupled to a distal portion of the main housing and adapted to frictionally engage and retract tissue(s) from a surgical site.

9. The surgical retractor of claim 8, wherein the one or more belts are manually operated.

10. The surgical retractor of claim 8, wherein one or more belts have a plurality of grooves adapted to securely grasp tissue.

11. The surgical retractor of claim 8, wherein one or more belts are comprised of a rubber-type material.

12. The surgical retractor of claim 8, wherein the handle is detachable from the main housing.

13. The surgical retractor of claim 8, wherein the main housing includes a lip at the distal-most portion for engaging tissue along an incision.

14. A method for retracting tissue, the method comprising:
providing a surgical retractor, the retractor including:
a main housing;
a handle coupled to a proximal portion of the main housing; and
one or more movable belts coupled to a distal portion of the main housing and adapted to frictionally engage tissue(s) adjacent to a surgical site; and
retracting the tissue(s) by moving the one or more of the movable belts.

15. The method of claim 14, including manually moving one or more of the belts.
